(19) United States
(12) Patent Application Publication
(10) Pub. No.: US 2026/0232989 A1
(43) Pub. Date: Aug. 13, 2026

(54) ROTOR, MAGNETIC COUPLING DEVICE, ELECTRIC MOTOR FOR A CARDIAC SUPPORT SYSTEM, PUMP UNIT FOR A CARDIAC SUPPORT SYSTEM, AND METHOD FOR PRODUCING A ROTOR

(71) Applicant: KARDION GMBH, Stuttgart (DE)

(72) Inventor: Uwe Vollmer, Sindelfingen (DE)

(21) Appl. No.: 19/633,467

(22) Filed: Mar. 30, 2026

Related U.S. Application Data
(63) Continuation of application No. 17/055,023, filed on May 16, 2019, now Pat. No. 12,589,238.

(30) Foreign Application Priority Data
May 16, 2018 (DE) 10 2018 207 594.8

(51) Int. Cl. A61M 60/492; A61M 60/216; A61M 60/419; A61M 60/804

(52) U.S. Cl. CPC A61M 60/492; A61M 60/216; A61M 60/419; A61M 60/804

(57) ABSTRACT

A rotor for a cardiac support system is disclosed. The rotor is assembled or can be assembled from at least four shell elements to form a hollow cylinder and/or on a shaft, wherein the shell elements are magnetized or can be magnetized alternately in magnetization direction which are oppositely directed or are orthogonal, so as to form a magnetized body having at least four magnetic poles.

### INCORPORATION BY REFERENCE TO ANY PRIORITY APPLICATIONS

The present application is a continuation of U.S. application Ser. No. 17/055,023 entitled “ROTOR, MAGNETIC COUPLING DEVICE, ELECTRIC MOTOR FOR A CARDIAC SUPPORT SYSTEM, PUMP UNIT FOR A CARDIAC SUPPORT SYSTEM, AND METHOD FOR PRODUCING A ROTOR” filed Jun. 11, 2021, which claims priority to PCT/EP 2019/062745 filed May 16, 2019, which claims priority to DE 102018207594.8 filed May 16, 2018. All of the above-listed applications and any and all other applications for which a foreign or domestic priority claim is identified in the Application Data Sheet as filed with the present application, are hereby incorporated by reference under 37 CFR 1.57, and form a part of this specification for all purposes.

### BACKGROUND

### Field

The invention relates to a rotor, a magnetic coupling device, an electric motor for a cardiac support system, a pump unit for a cardiac support system, and a method for producing a rotor.

### Description of the Related Art

Conventional magnetic coupling elements often have limitations with respect to magnetization density. This can lead to unfavorable magnetic coupling behavior and, in particular in application scenarios in which the available installation space is small, such as in medical devices that are to be implanted in the body of a patient, makes said elements disadvantageous.

### SUMMARY

The object of the invention is to create a rotor which can be used in a magnetic coupling device, in an electric motor for a cardiac support system and/or in a pump unit for a cardiac support system, and which enables the provision and/or transmission of high torques in a small installation space.

This object is achieved by the rotor described herein. Advantageous embodiments of the invention are described herein.

The rotor according to the invention and described herein is in particular suitable for use in a cardiac support system. A rotor according to the invention is or can be assembled from at least four shell elements to form a hollow cylinder and/or on a shaft, wherein the shell elements are or can be magnetized alternately in oppositely directed or orthogonal magnetization directions in order to form a magnetic body having at least four magnetic poles.

Shell elements in the present case can, for example, be permanent magnets or ferromagnetic or magnetizable elements which are or can be magnetized in a predetermined magnetization direction. The shell elements here are magnetized, for example, before they are assembled to form the rotor. The shell elements can furthermore be disposed such that adjacent shell elements are magnetized in oppositely directed or orthogonal magnetization directions.

The invention is based on the knowledge that a very high magnetization density and thus a high magnetic flux can be achieved, because a rotor can be formed by using shell elements that are disposed in such a way that adjacent shell elements are poled in oppositely aligned or orthogonal magnetization directions. In this way, a high magnetic flux can be ensured on the surface of the rotor, which can then be used to achieve a high magnetic frictional connection to a further magnetic element opposite to the rotor, so that, for example, a magnetic coupling device capable of transmitting high torques can be created.

One advantageous embodiment of the invention provides that, in the assembled state of the rotor, a first shell element and a second shell element are disposed opposite one another to form a first pole pair, and a third shell element and a fourth shell element are disposed opposite one another to form a second pole pair. The advantage of this is that opposite shell elements form a respective pair of poles, so that a high magnetic flux through the rotor and over a surface of the rotor can be produced.

In a further advantageous embodiment of the invention, the first shell element is or can be parallel magnetized in a first magnetization direction, the second shell element is or can be parallel magnetized in a second magnetization direction opposite to the first magnetization direction, the third shell element is or can be parallel magnetized in a third magnetization direction orthogonal to the first magnetization direction and the second magnetization direction and the fourth shell element is or can be parallel magnetized in a fourth magnetization direction opposite to the third magnetization direction. The advantage of such an embodiment is that a very high magnetic flux density can be realized on an outer surface of the rotor, so that high torque transmission is made possible with such a rotor. The anisotropy of the material of the shell elements with respect to the magnetization can thus be compensated very efficiently.

An embodiment of the invention in which the shell elements are or can be radially magnetized has a particularly high magnetic flux density. The advantage of such an embodiment is that the magnetic flux lines of the shell elements emerge substantially perpendicularly on an outer surface of the rotor, so that a homogeneous, radial magnetic field can be produced around the rotor, which enables a favorable and efficient transmission of torque.

According to a further embodiment of the approach proposed here, the rotor can also comprise a shaft, whereby the shaft and the magnetic body are or can be connected to one another in a torsionally rigid manner. The magnetic body can be glued or pressed onto the shaft, for example. Such an embodiment permits cost-effective production of a magnetically very effective rotor.

An embodiment of the invention in which the rotor has a diameter less than 20 mm, in particular less than 10 mm, is particularly efficient. The advantage of such an embodiment is that, on the one hand, such a rotor can be manufactured very easily and, on the other hand, it enables a very high transmission of torque compared to known magnetic coupling elements or rotors.

A magnetic coupling device according to the invention has the following features:
a rotor according to a variant presented here; and
a further magnetic body, which is or can be assembled from at least four further shell elements to form a hollow cylinder, wherein the further shell elements are or can be magnetized in oppositely directed magnetization directions to form at least four further magnetic poles, wherein the magnetic body of the rotor and the further magnetic body are or can be disposed one inside the other, in particular are or can be disposed concentrically to one another, and are or can be mounted so as to be rotatable relative to one another.

Such a magnetic coupling device enables very efficient transmission of torque, in particular in very small installation spaces, by producing a high flux density between the rotor and the further magnetic body.

According to a preferred embodiment of the invention, the magnetic body and the further magnetic body can in particular each have the same number of magnetic poles. This enables particularly efficient guidance of the magnetic field, which is characterized by a high ability to transmit torque.

According to a further embodiment of the invention, the further magnetic body is or can also be assembled from at least four additional shell elements to form the hollow body, wherein, in the assembled state of the further magnetic body, each one of the four additional shell elements is respectively disposed between two of the four further shell elements, wherein each of the four additional shell elements is or can be magnetized in tangential direction, for example to form a Halbach-Array. Such an embodiment of the approach proposed here offers the advantage that the additional shell elements, each of which is or can be magnetized in tangential direction, enable efficient guidance of magnetic lines in the further magnetic body in order to, if possible, prevent the magnetic field lines from escaping from the further magnetic body. It is thus possible to ensure a high magnetic flux between the rotor and the further magnetic body formed as a hollow cylinder, which consequently again enables a very efficient transmission of torque between the rotor and the further magnetic body or the hollow body.

The invention also extends to an electric motor comprising a rotor according to a variant presented here and/or a magnetic coupling device according to a variant presented here. The aforementioned advantages can be realized or implemented very efficiently with such an embodiment as well.

The invention in particular extends to a pump unit for a cardiac support system, wherein the pump unit comprises a rotor according to a variant presented here and/or a magnetic coupling device according to a variant presented here. Such a pump unit for a cardiac support system in particular requires high torque transmission in a small available installation space, so that the approach presented here can be used particularly favorably in such an application scenario in a patient.

The invention furthermore also extends to a method for producing a rotor having the features specified above, wherein the method comprises the following steps:
Assembling at least four shell elements alternately in oppositely directed or orthogonal magnetization directions to form a hollow cylinder and/or on a shaft, in order to form a magnetic body having at least four magnetic poles, in order to produce the rotor.

Such an embodiment, too, makes it possible to realize the aforementioned advantages in a technically simple, cost-effective and efficient manner.

This method can be implemented in software or hardware, for example, or in a combination of software and hardware, for example in a control device.

The approach presented here further creates a device which is configured to carry out, control or implement the steps of a variant of a method presented here in corresponding facilities. The underlying object of the invention can likewise be achieved quickly and efficiently using this design variant of the invention in the form of a device.

For this purpose, the device can comprise at least one computation unit for processing signals or data, at least one memory unit for storing signals or data, at least one interface to a sensor or an actuator for inputting sensor signals from the sensor or for outputting data or control signals to the actuator, and/or at least one communication interface for inputting or outputting data embedded in a communication protocol. The computation unit can be a signal processor, a microcontroller or the like, for example, whereas the memory unit can be a flash memory, an EPROM or a magnetic memory unit. The communication interface can be configured to input or output data in a wireless and/or a wired manner, whereby a communication interface that can input or output wired data can, for example, input said data electrically or optically from a corresponding data transmission line or output it into a corresponding data transmission line.

In the present case, a device can be an electrical device that processes sensor signals and outputs control and/or data signals as a function of said signals. The device can comprise an interface that can be hardware and/or software-based. In the case of a hardware-based configuration, the interfaces can be part of a so-called system ASIC, for example, which contains the various functions of the device. However, it is also possible for the interfaces to be separate, integrated circuits or consist at least in part of discrete components. In the case of a software-based configuration, the interfaces can be software modules that are, for example, provided on a microcontroller alongside other software modules.

A computer program product or computer program with program code, which may be stored on a machine-readable carrier or storage medium such as a semiconductor memory, a hard drive memory or an optical memory, and is used to carry out, implement and/or control the steps of the method according to one of the above-described embodiments, is also advantageous, in particular if the program product or program is executed on a computer or device.

### BRIEF DESCRIPTION OF THE DRAWINGS

Advantageous design examples of the invention are shown in the drawings and explained in more detail in the following description.

The figures show:

FIG. 1A a cross-sectional illustration of a rotor which is disposed on or glued onto a shaft of a motor;

FIG. 1B a side view of the rotor which is disposed on or glued onto the shaft;

FIG. 2 a cross-sectional illustration of a rotor which is disposed on or glued onto a shaft of a motor, whereby a four-poled magnet is now formed as a rotor by impressing the magnetic field lines;

FIG. 3A a cross-sectional illustration of a rotor according to a design example of the present invention;

FIG. 3B a side view of the rotor of FIG. 3A which is disposed on or glued onto the shaft;

FIG. 4 a cross-sectional illustration of a rotor according to a further design example of the present invention;

FIG. 5 a cross-sectional illustration of a magnetic coupling device according to a further design example of the present invention comprising a rotor;

FIG. 6 a schematic illustration of a patient implanted with a cardiac support system comprising a magnetic coupling device or a rotor according to a design example of the invention; and

FIG. 7 a flow diagram of a method for producing a rotor according to a design example.

### DETAILED DESCRIPTION

In the following description of favorable design examples of the present invention, the same or similar reference signs are used for elements shown in the various figures and having a similar effect; a repeated description of these elements is omitted.

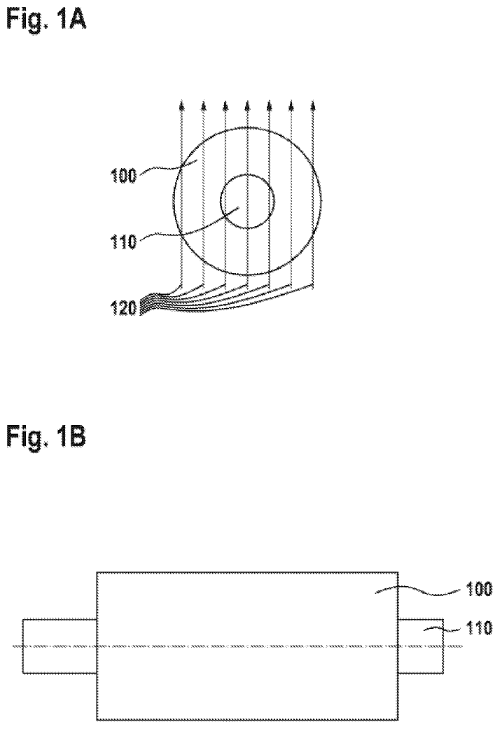

FIG. 1A shows a cross-sectional illustration of a rotor 100, which is disposed on or glued onto a shaft 110 of a motor. The rotor 100 here is configured as a hollow cylinder. Also shown are magnetic field lines 120 which act on the magnetizable element(s) of the rotor 100 in order to magnetize the rotor. The magnetic field lines, which have been provided with the reference sign 120, can also be understood as magnetization directions at the respective positions on the rotor 100.

FIG. 1B shows a side view of the rotor 100, which is disposed on or glued onto the shaft 110. The rotor 100 can have a magnet length of 10 mm, a magnet height of 1 mm and/or a shaft diameter of 1 mm, for example. The rotor 100 can, for example, be used as a component of a magnetic coupling of a cardiac support system to make the best possible use of the available installation space in the patient's body or artery.

Magnetic couplings and electric motors having very small diameters in the range of less than approximately 20 mm use axles (such as the shaft 110 here in FIGS. 1A and 1B) having permanent magnets attached to the axle, the so-called rotors 100, which can also comprise further structural elements such as magnetic returns (yokes) between the shaft or bearings pressed onto the shaft.

Due to the required or desired performance, these permanent magnets are preferably made of magnetic materials having a very high energy density. One characteristic of these magnetic materials is the strong anisotropy of the magnets with respect to the magnetization direction. This is used to make the best possible use of the material with respect to the achievable energy density in the magnet or to achieve the highest possible magnetic flux density in the magnetic circuit of the electric motors or magnetic couplings.

The manufacturing processes for permanent magnets having a very high energy density result in relatively large tolerances in the magnet dimensions. In the case of very small magnet dimensions in the rotors 100, this leads to relatively large tolerances in the components having the magnets. For this reason, the rotors 100 of small motors or couplings are preferably made of hollow-cylindrical permanent magnets, which are pushed and glued directly onto the non-magnetic or soft magnetic shaft. For the required high energy densities and two-pole couplings or electric motors, the initial magnetization process is performed diametrically across the entire cylinder in order to make optimum use of the anisotropy of the magnetic material, as shown in FIG. 1A.

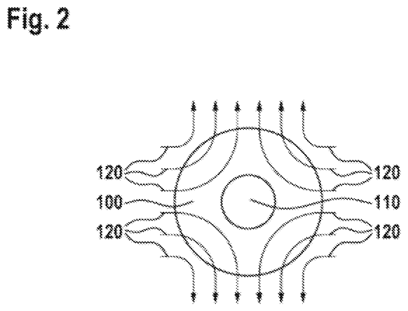

FIG. 2 shows a cross-sectional illustration of a rotor 100, which is disposed on or glued onto a shaft 110 of a motor, whereby a four-poled magnet is now formed as the rotor 100 by impressing the magnetic field lines 120 according to the illustration of FIG. 2. Therefore, a four-poled magnetic field is used to magnetize hollow-cylindrical permanent magnets. A magnetization for impressing 4 poles, as shown in FIG. 2, in such anisotropically prepressed magnet hollow cylinders leads to an uneven magnetic flux in the preferably orthogonal magnetization directions. This is not advantageous for the mentioned components, because the magnetic material is not fully utilized. If magnets without a preferred direction are used, owing to the principle involved only a comparatively lower energy density can be achieved, and consequently a smaller magnetic flux in the magnetic circuit of a coupling or an electric motor. This applies accordingly to higher pole numbers. In the typical design and manufacturing method, the available torque for both components drops sharply as a result of the small rotor diameter. Under the same boundary conditions (drive voltage and drive current), the maximum speed of electric motors can then become very high. For applications in which the focus is not so much on very high speeds, in which a high torque is more important, gears can be added to the drive train to shift the mechanical performance from high speeds and low torques to lower speeds and higher torques. This requires at least one further component and additional installation space. In certain applications, both implications are undesirable or simply not possible. This is the case for a left ventricular cardiac support system, which is placed in the aorta, for example, or completely or partially in the left ventricle.

To increase the performance of such coupling elements, it is useful to create a greater magnetic flux in the magnetic circuit. In many applications it is expedient to do this by achieving a higher torque. Advantageously, the number of pole pairs can be increased in order to achieve higher torques.

Since, as described above, this is not or only conditionally possible for hollow-cylindrical magnets at the required high energy densities and small dimensions, the proposal here, according to a design example of the invention, is to produce the required magnetic flux using magnetic shells, which are for example also segmented in tangential direction, as the shell elements that can be glued onto a shaft or a magnetic yoke.

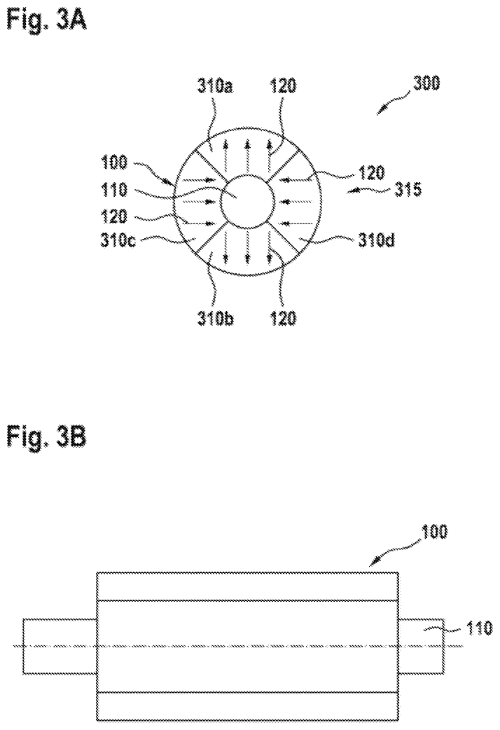

FIG. 3A shows a cross-sectional illustration of a magnetic coupling element 300 having a rotor 100 according to a design example of the present invention. The rotor 100 is formed here by at least four shell elements 310a, 310b, 310c and 310d, which are assembled to form a magnetic body 315, for example in the form of a hollow cylinder, and/or to be fixed on the shaft 110 in a non-rotatable manner. The shell elements 310a-d are disposed such that the individual shell elements 310a-d are or can be magnetized alternately in opposite or orthogonal directions. For this purpose, the shell elements 310a, 310b, 310c and 310d are made of a hard magnetic or ferromagnetic material or are already directly magnetized as permanent magnets. In FIG. 3A, this alignment of the magnetization is illustrated by the schematically drawn magnetic field lines 120, which now correspond to the magnetic field lines 120 caused by a magnetic field produced by the respective shell elements 310 themselves as permanent magnets. FIG. 3A shows, in particular, that the first shell element 310a is polarized such that the magnetic field lines 120 of this shell element 310a point upward, whereas the magnetic field lines 120 of the third shell element 310c, which adjoins the first shell element 310a on the left, point to the right and are thus aligned orthogonally to the magnetic field lines 120 of the first shell element 310a. The magnetic field lines 120 of the second shell element 310b, which adjoins the third shell element 310c at the bottom, point downward and thus in a direction opposite to the direction of the magnetic field lines 120 of the first shell element 310a. The magnetic field lines 120 of the fourth shell element 310d, which adjoins the second shell element 310a on the right, are directed to the left and thus orthogonal to the magnetic field lines 120 of the first and second shell element 310a and 310b. The magnetic field lines 120 of the fourth shell element 310d point in a direction opposite to the direction of the magnetic field lines 120 of the third shell element 310c. Thus, FIG. 3A shows a four-poled rotor 100 comprising shell elements 310 or shell magnets with parallel magnetization which are disposed or mounted on the shaft 110.

FIG. 3B shows a side view of the rotor 100 of FIG. 3A, which is disposed on or glued onto the shaft 110. The rotor 100 can again have a magnet length (shell element length) of 10 mm, a magnet height of 1 mm and/or a shaft diameter of 1 mm, for example. The rotor 100 can, for example, be used as a component of a magnetic coupling of a cardiac support system to make the best possible use of the available installation space in the patient's body or artery.

In the approach shown in FIGS. 3A and 3B, the shell elements 310a to 310d, which, in terms of dimensions, are identical magnetic shells for example, are preferably magnetized in parallel, for example alternately in radially positive or radially negative direction. A given anisotropy of the magnetic shells or shell elements 310a to 310d can thus be optimally utilized to maximize the magnetic flux in the magnetic circuit.

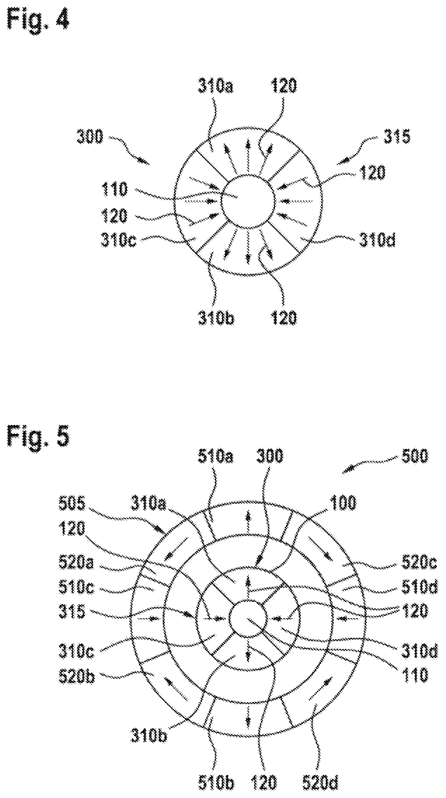

FIG. 4 shows a cross-sectional illustration of a magnetic coupling element 300 having a rotor 100 according to a further design example of the present invention. It is thus possible to realize a radial magnetization, but in which, with the same dimensions, a slightly smaller magnetic flux occurs in the magnetic flux compared to the magnetization according to the illustration of FIG. 3A. In such a radial magnetization, the magnetic field lines 120 are aligned radially toward the shaft 110 or away from the shaft 110, so that there are no parallel magnetic field lines 120. Rather, the magnetic field lines 120 enter or exit perpendicularly from a surface of the shell elements 310a to 310d. Such a magnetization can also be produced in a technically simple manner, for example via a core as a shaft 110 which is used to magnetize the shell elements 310a [sic] to 310d. In this design example, the magnetic field lines 120 of adjacent shell elements 310a to 310d furthermore extend in an opposite direction, for example, whereas the magnetic field lines 120 of adjacent shell elements 310a to 310d are aligned orthogonally to one another as shown in the illustration of FIG. 3A.

The number of pole pairs of a rotor 100 or a magnetic body can be two or more. By using magnetic materials having the smallest possible grain size and, if necessary, reworking, the loss of magnet mass can be kept small while maintaining the same external dimensions of the components or shell elements 310. In rotors 110 having pole pair numbers greater than one, the amount of energy for torque transmission, which is in principle lower due to the smaller magnet mass resulting from the tolerances, is more than compensated by the better utilization of the magnetic material due to the use of anisotropic material.

FIG. 5 shows a cross-sectional illustration of a magnetic coupling device 500 according to a further design example of the present invention comprising a magnetic coupling element 300 having a rotor 100 as described above, for example. In this magnetic coupling element 300, the shell elements 310a to 310d of the rotor 100 are formed according to the illustration of FIG. 3A; i.e. with adjacent shell elements 310 comprising magnetic field lines 120 which aligned orthogonally to one another. The magnetic coupling device 500 also comprises a further magnetic body 505, which is hollow-cylindrical, for example, and comprises at least four further shell elements 510a, 510b, 510c and 510d. The further shell elements 510a to 510d are or can be magnetized in oppositely directed magnetization directions corresponding to the magnetic field lines 120 in order to form at least four further magnetic poles. Furthermore, the magnetic body of the rotor 100 and the further magnetic body 500 are or can be disposed one inside the other, in particular are or can be disposed concentrically to one another, and are or can be mounted so as to be rotatable relative to one another. An orientation of the magnetic field lines 120 of the further shell elements 510a to 510d can correspond to the orientation of the magnetic field lines 120 of the shell elements 310a to 310d. For example, the orientation of the magnetization of the further shell elements 510a to 510b can correspond to the orientation of the shell elements 310a to 310d according to the illustration of FIG. 3A.

It is thus possible to realize a magnetic coupling device 500, which enables a very efficient utilization of the magnetization of the materials for the rotor 100 and a hollow cylinder as a further magnetic body 505 and thus opens up an efficient torque transmission of a torque from the shaft 110 to the hollow cylinder.

A design example of the approach presented here, which, as shown in FIG. 5, comprises four additional shell elements 520a, 520b, 520c and 520d, is particularly advantageous. In the assembled state of the further magnetic body 505, each one of these four additional shell elements 520a, 520b, 520c, 520d is respectively disposed between two of the four additional shell elements 510a to 510d, wherein each of the four additional shell elements 520a to 520d is or can be magnetized in tangential direction. A shell element 520a to 520d can be considered to be a shell element 520a to 520d magnetized in tangential direction if the magnetization, or a magnetic field line 120 caused by such magnetization, of said shell element 520a to 520d is aligned tangentially to an outer surface with respect to a further magnetic body 505 configured as a hollow cylinder. It is thus now possible to guide a magnetic flux in the further magnetic body 505 such that a high flux density of the magnetic field between the further magnetic body 505 and the magnetic coupling element 300 or rotor 100 can be achieved, which in turn leads to an efficient coupling of the further magnetic body 505 and the rotor 110 or the magnetic coupling element. Thus, a tangentially segmented, multi-pole magnetic axis for miniaturized magnetic couplings and electric motors is presented here as well, in particular for use in a pump of a cardiac support system.

FIG. 5 therefore shows an outer magnet ring having the same number of pole pairs as in the rotor 100, here as a design with a Halbach array. In principle, with the higher pole pair number, the thus increased magnetic flux in the magnetic circuit produces a greater torque in said components compared to two-poled rotors or multi-poled rotors made of hollow-cylindrical magnets. In electric motors, the winding should be adapted accordingly to accommodate the higher number of pole pairs. In radial magnetic couplings, the outer hollow-cylindrical magnet must also be adapted to the pole number. The outer hollow cylinder can optionally consist of a solid cylinder, joined magnetic shells corresponding to the magnetic shells on the rotor 110, or a Halbach array as shown in FIG. 5.

Such a rotor design can advantageously be used, in particular in left ventricular cardiac support systems (LVAD). In principle, however, all applications requiring high power density and/or high torque are predestined for this type of rotor design.

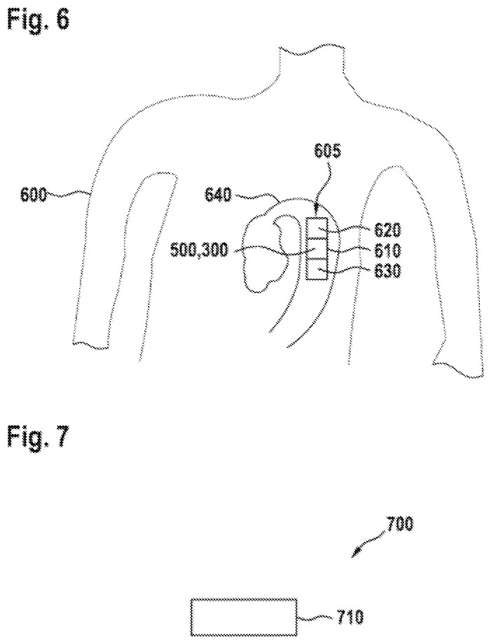

FIG. 6 shows an illustration of a patient 600 implanted with a cardiac support system 605. The cardiac support system 605 includes a pump unit 610, which comprises a magnetic coupling device 500 for coupling an electric motor 620 to an impeller 630 for conveying blood of the patient 600 and wherein the pump unit 610 is implanted, for example in an aorta 640 of the patient 600.

FIG. 7 shows a flow diagram of a method 700 for producing a rotor according to a design example described here, wherein the method 700 includes a step 710 of assembling at least four shell elements alternately in oppositely directed or orthogonal magnetization directions to form a hollow cylinder and/or on a shaft, in order to form a magnetic body having at least four magnetic poles, in order to produce the rotor.

If a design example includes an “and/or” conjunction between a first feature and a second feature, this should be read to mean that the design example according to one embodiment comprises both the first feature and the second feature and, according to another embodiment, comprises either only the first feature or only the second feature.

1. (canceled)

2. A pump unit for a cardiac support system, the pump unit comprising:
an electric motor comprising a shaft;
an impeller configured to convey blood of a patient; and
a magnetic coupling device configured to couple the electric motor to the impeller comprising:
a rotor comprising a first magnetic body disposed on the shaft, the first magnetic body comprising a first plurality of shell elements, wherein a first subset of the first plurality of shell elements are magnetized in radially opposite magnetization directions, and the first magnetic body comprising at least four magnetic poles; and
a second magnetic body comprising a second plurality of shell elements,
wherein the second magnetic body is coupled to the impeller so that rotation of the rotor drives rotation of the impeller,
wherein a first subset of the second plurality of shell elements are magnetized in opposing directions to form at least four magnetic poles,
wherein the first magnetic body is positioned concentrically inside the second magnetic body.

3. The pump unit of claim 2, wherein the first magnetic body and the shaft are connected to one another in a torsionally rigid manner.

4. The pump unit of claim 2, wherein the first magnetic body and the second magnetic body are rotatable relative to one another.

5. The pump unit of claim 2, wherein a second subset of the second plurality of shell elements are magnetized in directions tangential to an outer surface of the second magnetic body.

6. The pump unit of claim 5, wherein, each shell element of the second subset of the second plurality of shell elements is positioned between two adjacent shell elements of the first subset of the second plurality of shell elements.

7. The pump unit of claim 2, wherein the first plurality of shell elements are configured to form a hollow cylinder.

8. The pump unit of claim 2, wherein the first plurality of shell elements are permanent magnets.

9. The pump unit of claim 2, wherein the first plurality of shell elements comprises a first shell element, a second shell element, a third shell element, and a fourth shell element, and wherein the first shell element and the second shell element are arranged opposite to one another to form a first pole pair, and the third shell element and the fourth shell element are arranged opposite to one another to form a second pole pair.

10. The pump unit of claim 9, wherein:
the first shell element is magnetized in a first magnetization direction;
the second shell element is magnetized in a second magnetization direction opposite to the first magnetization direction;
the third shell element is magnetized in a third magnetization direction orthogonal to the first magnetization direction and the second magnetization direction; and
the fourth shell element is magnetized in a fourth magnetization direction opposite to the third magnetization direction.

11. The pump unit of claim 2, wherein the rotor has a diameter smaller than 20 mm.

12. The pump unit of claim 2, wherein the electric motor further comprises a winding adapted to the at least four magnetic poles of the first magnetic body.

13. The pump unit of claim 2, wherein the second magnetic body comprises a magnetic ring configured as a Halbach array.

14. The pump unit of claim 2, wherein the second plurality of shell elements are configured to form a hollow cylinder.

15. The pump unit of claim 2, wherein the first magnetic body and the second magnetic body each comprise the same number of magnetic poles.

16. The pump unit of claim 2, wherein the first plurality of shell elements are glued directly onto the shaft.

17. The pump unit of claim 2, wherein the first plurality of shell elements are comprised of a ferromagnetic material.

18. The pump unit of claim 2, wherein the rotor has a diameter of less than 20 mm.

19. The pump unit of claim 2, wherein at least one of the first plurality of shell elements has a length of 10 mm and a height of 1 mm.

20. The pump unit of claim 2, wherein the shaft has a diameter of 1 mm.

21. The pump unit of claim 2, wherein the shaft is a magnetic yoke.
